(19) United States
(12) Patent Application Publication
(10) Pub. No.: US 2025/0194867 A1
(43) Pub. Date: Jun. 19, 2025

(54) SPONGE ADAPTED FOR CLEANSING OF EARS AND FACE

(71) Applicants: Symeon Raptis, Attica (GR); Marinos Kontaras, Attica (GR)

(72) Inventors: Symeon Raptis, Attica (GR); Marinos Kontaras, Attica (GR)

(21) Appl. No.: 18/609,423

(22) Filed: Mar. 19, 2024

Related U.S. Application Data
(60) Provisional application No. 63/611,484, filed on Dec. 18, 2023.

(51) Int. Cl. A47K 7/02; A61F 11/00

(52) U.S. Cl. CPC A47K 7/02; A61F 11/006

(57) ABSTRACT

According to an aspect of the present invention, there is provided sponge adapted for cleansing of ears and facial structures, comprising: an inner facet of the sponge integrating a surface texture optimized for outer ear cleaning, exfoliation, and enhancing dermal microcirculation, wherein the inner facet has a bump configured to fit near an ear canal and a shape configured to surround an outer ear; and an outer layer having a shape configured for facial cleansing and cleaning the structures around facial structures, wherein: the inner facet and outer layer are combined together in a specific circular fashion, in which parts and edges of one component are pressed against specific parts of the other, forming a full facial sponge with only the outer sides of the components are visible and usable.

### BACKGROUND

Devices for cleaning ears and faces have been described in the prior art patent literature.

For example, U.S. Pat. No. 11,045,357 discloses an ear cleaning device for removing debris from an ear. In one form, the ear cleaning device includes a shaft, a head connected to the shaft, and a contiguous pocket structure of the head. The contiguous pocket structure includes a plurality of pockets that may be configured to receive debris. In another form, the ear cleaning device has a handle, a bulbous cleaner connected to the handle, and a solid inner core of the bulbous cleaner. The bulbous cleaner has two or more spaced, longitudinal walls disposed outward of the inner core that are configured to remove debris with turning of the ear cleaning device about a longitudinal axis of the device.

US20190290538 discloses an eyelid care appliance and method of using such appliance for preventing and treating blepharitis and mammalian meibomian gland dysfunction caused by gland obstruction. The method and apparatus of the invention enable restoration and maintenance of eyelid hygiene and the natural flow of secretions from the meibomian glands. A preferred embodiment of the apparatus comprises an oscillating spongehead affixed to the distal end of the eyelid care appliance that is equipped with a video camera, near field communications link, and a display, thereby enabling a user to “fly” the spongehead onto an eyelid margin for self-administered cleaning of the user's eyelid margins. The spongehead can be impregnated with topical pharmacologic or cleansing agents to better facilitate cleaning and therapeutic efficacy.

However, prior art devices for cleaning ears focus on the internal structures of the ears, and do not provide effective means of cleaning the exterior surfaces. What is needed is a simple and convenient device for cleaning exterior ears and facial structures.

### SUMMARY OF INVENTION

Therefore, the present invention is an ear and facial shower sponge, comprising two substantially mirrored components for the two sides of a user's face.

According to an aspect of the present invention, there is provided sponge adapted for cleansing of ears and faces, comprising: an inner facet of the sponge integrating a surface texture optimized for outer ear cleaning, exfoliation, and enhancing dermal microcirculation, wherein the inner facet has a bump configured to fit near an ear canal and a shape configured to surround an outer ear; and an outer layer having a shape configured for cleaning facial structures, wherein: the inner facet and outer layer are combined together in a specific circular fashion, in which parts and edges of one component are pressed against specific parts of the other, forming a full facial sponge with only the outer sides of the components are visible and usable.

### BRIEF DESCRIPTION OF THE DRAWINGS

FIG. 1 illustrates a sponge adapted for cleaning ears and other facial structures.

FIG. 2 illustrates two sponges adapted for simultaneous cleansing of two ears.

FIG. 3 illustrates an embodiment with teeth.

### DETAILED DESCRIPTION

Traditional bathing routines often involve using separate tools for facial cleansing and skin care routines, leading to time-consuming and sometimes cumbersome processes. The multi-purpose sponge of the present invention streamlines this by combining both functions into a single, easy-to-use tool, saving time and simplifying daily hygiene practices.

The present invention is a multi-purpose shower sponge designed for enhanced personal grooming and hygiene. It consists of two nearly identical components, each constructed from one or more different sponge materials which are securely bonded together. This innovative shower facial and ear sponge serves as a multifunctional tool with two distinct sides. The two components may consist of, without limitation, materials such as Konjac, Cellulose, Loofah or other additional variations.

The invention's design, with its specialized textures for facial use, that promotes an improved skin health. The gentle side is effective for facial cleansing without being harsh on the skin, while the textured side aids in massage and exfoliation, helping to maintain a healthy and clean area around the ears and the face.

The inner facets of the sponge integrate a surface texture optimized for outer ear cleaning, exfoliation, and enhancing dermal microcirculation. It further aids in the uniform dispersion of face cleansers. It is designed to adjust to a general ear type with suitable bumps and curves, to deeply clean the outer ear.

The outer layers incorporate a supple surface conducive to meticulous facial cleansing, expeditiously removing contaminants, cosmetics, and impurities while fostering cutaneous well-being.

The outer layers have a special shape which will be suitable for smoothly cleaning cheeks, such expeditiously removing makeup and cosmetics.

The two surfaces are combined together in a specific circular fashion, in which parts and edges of one component are pressed against specific parts of the other, forming a full facial sponge, as only the outer sides of the components are visible and usable in this situation.

Also, while the two components are combined, two of the side faces, with an optimized shape, may be suitable for smooth facial cleansing, such expeditiously removing makeup and cosmetics. That way of combining the two components allows a compact and flexible design for the final product.

The multi-functionality sponge is primarily intended for non-medical personal use, as well as potential application within the spa and wellness industry. It may be marketed as a standalone beauty and personal care product and is also well-suited for travel and on-the-go use.

For facial cleansing, the user may utilize the full sponge or the outer sides of the components separately. For ear cleansing, the user may utilize the inner sides of the components separately. Each component is intended for the corresponding ear, due to its optimized shape, so they can concentrate on cleaning both ears separately at the same time.

Left Component (L): One of the two nearly identical components of the sponge.

Right Component (R): The other of the two nearly identical components of the sponge.

Outer Layer-Facial Cleansing Surface (BL, BR): Each component (L and R) features an outer layer with four reference numerals (BL, BR). These surfaces have a soft and gentle texture suitable for facial cleansing, constructed from materials like Konjac, Cellulose, Loofah, or other variations as per the design.

Inner Layer-Ear Cleansing Surface (CL, CR): The inner layer of each component (L and R). These surfaces are optimized for ear cleansing, offering a textured design for the distribution of ear care products and scalp massage.

The Bonding Point is the specific circular movement where the two components (L and R) are securely bonded together. This bonding ensures the durability and integrity of the multi-purpose sponge, allowing for compact and flexible design. The overall thickness of each component, encompassing both layers and their materials. The length, width, and any specific contours or shapes for each component can vary. A user handle can also be provided.

Material Composition: the materials used for the outer and inner layers of each component and their properties, such as texture, softness, or exfoliating capabilities, can include a variety of foam, plastic, and other similar materials.

The sponge can be provided with perforations or channels to enhance water retention or product dispersion.

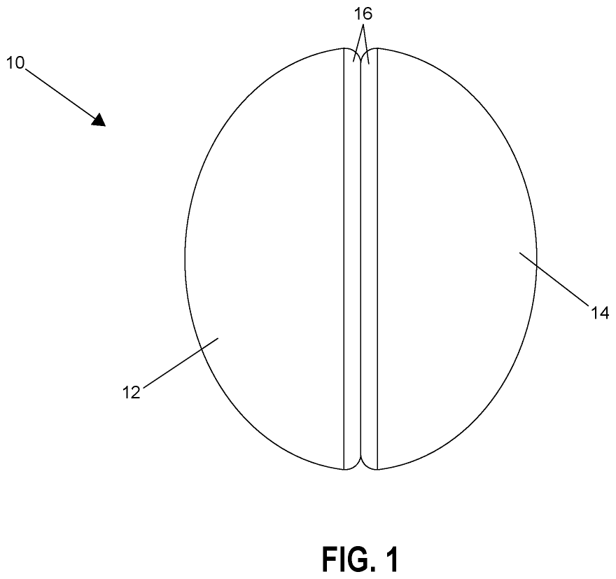

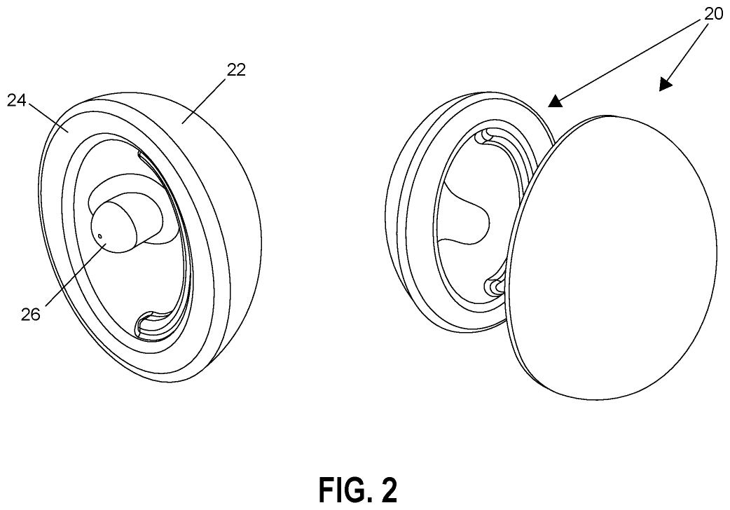

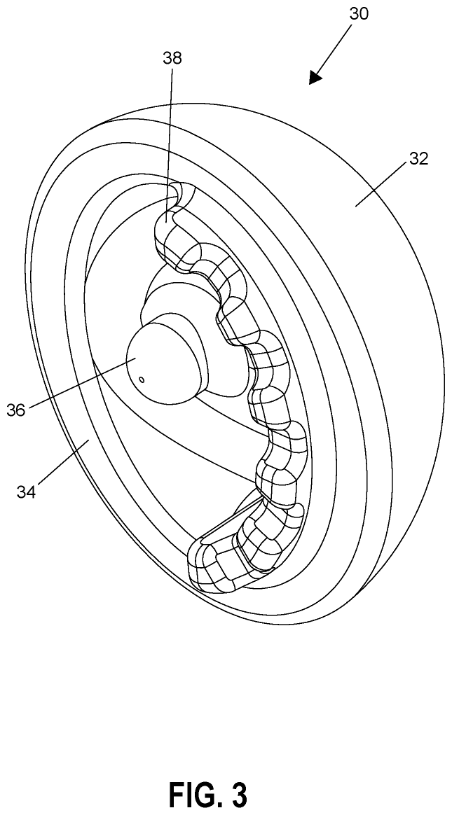

FIGS. 1, 2 and 3 illustrate the following components and features. FIG. 3 illustrates an embodiment with teeth.
10—device
12—left component
14—right component
16—bonding layer
20—inner structure of the device
22—outer casing
24—facial cleaning surface
26—ear cleansing surface
30—second embodiment
32—outer casing
34—facial cleaning surface
36—ear cleansing surface
38—teeth

The sponges are two equal parts with each part as described above with reference to FIG. 1. The right and left parts can be made by the same mold in the manufacturing, and provided as a system which can be used simultaneously.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Similar numerals designate similar elements among the several figures. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A sponge adapted for cleansing of ears and facial structures, comprising:
an inner facet of the sponge integrating a surface texture optimized for outer ear cleaning, exfoliation, and enhancing dermal microcirculation, wherein the inner facet has a bump configured to fit near an ear canal and a shape configured to surround an outer ear; and
an outer layer having a shape configured for cleaning facial structures and cleaning the structures around the face, wherein:
the inner facet and outer layer are combined together in a specific circular fashion, in which parts and edges of one component are pressed against specific parts of the other, forming a full facial sponge with only the outer sides of the components are visible and usable.

2. The sponge adapted for cleansing of ears and facial structures of claim 1, wherein the outer layer is comprised of one or more of Konjac, Cellulose, and Loofah.

3. A system, comprising two of the sponges of claim 1 packaged together.

4. A method, comprising simultaneously using the system of claim 2 to clean two ears at the same time.
